(19) United States
(12) Patent Application Publication
(10) Pub. No.: US 2021/0380596 A1
(43) Pub. Date: Dec. 9, 2021

(54) UPADACITINIB CRYSTAL FORM AND PREPARATION METHOD THEREFOR AND USE THEREOF

(71) Applicant: CRYSTAL PHARMACEUTICAL (SUZHOU) CO., LTD., Suzhou, Jiangsu (CN)

(72) Inventors: Minhua CHEN, Suzhou (CN); Jiaming SHI, Suzhou, Jiangsu (CN); Jing ZHANG, Suzhou (CN); Huize JING, Suzhou (CN)

(21) Appl. No.: 17/288,063

(22) PCT Filed: Feb. 29, 2020
(86) PCT No.: PCT/CN2020/077327

§ 371 Date: Apr. 23, 2021

(30) Foreign Application Priority Data
Mar. 1, 2019 (CN) 201910156955.9
Apr. 30, 2019 (CN) 201910358029.X

(51) Int. Cl. C07D 487/14

(52) U.S. Cl. CPC C07D 487/14; C07B 2200/13

(57) ABSTRACT

The present disclosure relates to a novel crystalline form of upadacitinib and processes for preparation thereof. The present disclosure also relates to pharmaceutical compositions containing the crystalline form, use of the upadacitinib crystalline form for preparing JAK inhibitor drug, and use of the upadacitinib crystalline form for preparing drugs treating rheumatoid arthritis. The crystalline form of upadacitinib provided by the present disclosure has one or more improved properties compared with prior art and has significant values for future drug optimization and development.

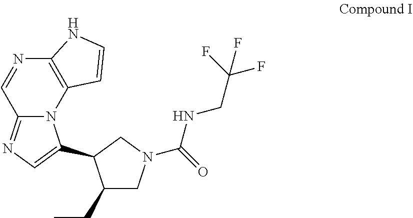 [SMILES: CC[C@@H]1CN(C(=O)NCC(F)(F)F)C[C@@H]1c1cnc2cnc3[nH]ccc3n12]

### TECHNICAL FIELD

The present disclosure relates to the field of pharmaceutical chemistry, particularly relates to a novel crystalline form of upadacitinib, processes for preparation and use thereof.

### BACKGROUND

Rheumatoid arthritis is an autoimmune disease that can cause chronic inflammation in joints and other parts of the body and leads to permanent joint damage and deformities. If not treated, rheumatoid arthritis can lead to substantial disability and pain due to the damage of joint function, which ultimately leads to shorter life expectancy. Crohn's disease is an inflammatory bowel disease. Symptoms usually include abdominal pain, diarrhea, fever, and weight loss. Those with the disease are at greater risk of colon cancer. Ulcerative colitis is a chronic disease that causes inflammation, ulcers of colon and rectum. The main symptoms are abdominal pain and diarrhea with bloody stools. The symptoms usually progress slowly and vary in severity. The common symptoms of atopic dermatitis include itchy, redness and inflamed, and cracked skin. Patients with atopic dermatitis may also have hay fever and asthma. Psoriatic arthritis is an inflammatory arthropathy associated with psoriasis, with a psoriasis rash and accompanied with pain, swelling, tenderness, stiffness, and movement disorders in the joints and surrounding soft tissues.

Janus kinase 1 (JAK1) is a target for immune-inflammatory diseases, and its inhibitors are beneficial for the treatment of immune-inflammatory disorders diseases, such as rheumatoid arthritis, Crohn's disease, ulcerative colitis, atopic dermatitis, psoriatic arthritis, etc.

Upadacitinib is a second-generation oral JAK1 inhibitor developed by AbbVie, with a high selectivity for JAK1. The chemical name of upadacitinib is: (3S,4R)-3-ethyl-4-(3H-imidazo[1,2-a]pyrrolo [2,3-e] pyrazin-8-yl)-N-(2,2,2-trifluoroethyl) pyrrolidine-1-carboxamide (hereinafter referred to as “Compound I”), and the structure is shown as follows:

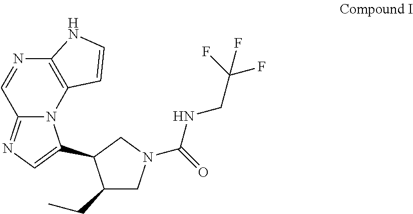 [SMILES: CC[C@@H]1CN(C(=O)NCC(F)(F)F)C[C@@H]1c1cnc2cnc3[nH]ccc3n12]

A crystal is a solid material whose constituents are arranged in a microscopic structure with regular three-dimensional pattern. Polymorphism is the ability of a compound to exist in two or more crystalline forms. Different crystalline forms have different physicochemical properties and different in vivo dissolution and absorption, which will further affect drug's clinical efficacy and safety to some extent. In particular, for poorly soluble drugs, the above effects of the crystalline form will be greater. Therefore, drug polymorphism is an important part of drug research and an important part of drug quality control.

WO2017066775A1 disclosed upadacitinib freebase Form A, Form B, Form C, Form D, amorphous and salts thereof. This patent application disclosed that Form A and Form B have poor crystallinity and stability and can be easily dehydrated to form amorphous. Form D can only be obtained at low water activity. In addition, Form D crystallizes slowly and the process can hardly be repeated. Form D will convert to Form C at high water activity. Compared with other forms of upadacitinib free form disclosed in WO2017066775A1, Form C has better properties. However, it has disadvantages of poor repeatability and difficulty in crystallizing from solution.

As the molecules in the amorphous solids are randomly arranged, they are in a thermodynamically unstable state. Amorphous solids are in a high-energy state, and usually have poor stability. During the manufacturing and storage process, amorphous drugs are prone to crystal transformation, which leads to the change of drug bioavailability, dissolution rate, etc., resulting in changes in the drug's clinical efficacy. In addition, amorphous is usually prepared through a rapid kinetic precipitation process, which easily leads to excessive residual solvent. The particle property of amorphous is difficult to control in the preparation process, making it a great challenge in the practical application of drugs.

In order to overcome the disadvantages of the prior art, it is still needed to develop a crystalline form with good stability, good repeatability, easiness to be crystallized from solution and other properties meeting the pharmaceutical demands for the development of drugs containing upadacitinib. The inventors of the present disclosure surprisingly discovered crystalline form CSII of upadacitinib, which has advantages in at least one aspect of stability, melting point, solubility, in vitro and in vivo dissolution, hygroscopicity, bioavailability, adhesiveness, compressibility, flowability, processability, purification ability, and formulation development, etc. In particular, crystalline form CSII has advantages in solubility, stability, particle size distribution, compressibility, yield, flowability and adhesiveness, which provides a new and better choice for the development of upadacitinib and is of great significance.

### SUMMARY OF THE INVENTION

The main objective of the present disclosure is to provide a novel crystalline form of upadacitinib, processes for preparation and use thereof.

According to the objective of the present disclosure, crystalline form CSII of Compound I is provided (hereinafter referred to as Form CSII).

According to one aspect of the present disclosure, the X-ray powder diffraction pattern of Form CSII shows characteristic peaks at 2theta values of 20.2°±0.2°, 25.1°±0.2° and 27.7°±0.2° using CuKα radiation.

Furthermore, the X-ray powder diffraction pattern of Form CSII shows one or two or three characteristic peaks at 2theta values of 8.0°±0.2°, 23.0°±0.2° and 23.8°±0.2°. Preferably, the X-ray powder diffraction pattern of Form CSII shows three characteristic peaks at 2theta values of 8.0°±0.2°, 23.0°±0.2° and 23.8°±0.2°.

Furthermore, the X-ray powder diffraction pattern of Form CSII shows one or two characteristic peaks at 2theta values of 21.3°±0.2° and 12.1°±0.2°. Preferably, the X-ray powder diffraction pattern of Form CSII shows two characteristic peaks at 2theta values of 21.3°±0.2° and 12.1°±0.2°.

According to another aspect of the present disclosure, the X-ray powder diffraction pattern of Form CSII shows three or four or five or six or seven or eight or nine characteristic peaks at 2theta values of 4.0±0.2°, 20.2°±0.2°, 25.1°±0.2°, 27.7°±0.2°, 8.0°±0.2°, 23.0°±0.2°, 23.8°±0.2°, 21.3°±0.2° and 12.1°±0.2° using CuKα radiation.

Without any limitation being implied, the X-ray powder diffraction pattern of Form CSII is substantially as depicted in FIG. 1.

Without any limitation being implied, the thermo gravimetric analysis curve of Form CSII is substantially as depicted in FIG. 2, which shows 0.2%-1.4% weight loss when heated to 189° C. Without any limitation being implied, the differential scanning calorimetry curve of Form CSII is substantially as depicted in FIG. 3. An endothermic peak is at around 192-202° C., which is a melting endothermic peak.

Without any limitation being implied, Form CSII is an anhydrate.

According to the objective of the present disclosure, a process for preparing Form CSII is also provided, wherein the process comprises: Dispersing upadacitinib free base in an ether solvent, keeping the system at 10-100° C. for reaction to obtain crystalline form CSII.

Furthermore, said ether is R1-O—R2 or a mixture thereof. R1 and R2 are C2-C5 short-chain alkyls. preferably, said ether is isopropyl ether.

Furthermore, the time of said reaction is preferably 2-6 days, more preferably 4-5 days.

Furthermore, the temperature of said reaction is preferably 50-80° C.

Form CSII of the present disclosure has the following advantages:

(1) Compared with prior art, Form CSII has higher solubility. Compared with prior art, Form CSII has a higher solubility in pH7.4 PBS (Phosphate-buffered saline), FaSSIF (fasted state simulated intestinal fluids) and FeSSIF (fed state simulated intestinal fluids). In particular, the solubility of Form CSII is more than three times that of Form C in WO2017066775A1 in PBS and FaSSIF.

Higher solubility is beneficial to improve drug's in vivo absorption and bioavailability, thus improving drug efficacy. In addition, drug dose reduction without affecting efficacy is possible due to higher solubility, thereby reducing the drug's side effects and improving drug safety.

(2) Form CSII drug substance of the present disclosure has good stability. Crystalline state of form CSII drug substance doesn't change for at least three months when stored under the condition of 25° C./60% RH (relative humidity). The chemical purity is above 99% and remains substantially unchanged during storage. These results show that Form CSII drug substance of the present disclosure has good stability under long-term condition, which is suitable for drug product storage. After Form CSII is mixed with the excipients to form a drug product, and stored under the condition of 25° C./60% RH, crystalline state of Form CSII drug product doesn't change for at least three months. The chemical purity of the drug substance in drug product is above 99% and remains substantially unchanged during storage. These results show that Form CSII drug substance and drug product of the present disclosure have good stability under long-term condition, which is suitable for drug product storage.

Meanwhile, crystalline state of Form CSII drug substance doesn't change for at least three months when stored under the condition of 40° C./75% RH. Crystalline state of Form CSII drug substance doesn't change for at least one month when stored under the condition of 60° C./75% RH. The chemical purity is above 99% and remains substantially unchanged during storage. After Form CSII is mixed with the excipients to form a drug product and stored under the condition of 40° C./75% RH, crystalline state of Form CSII drug product doesn't change for at least three months. The chemical purity of the drug substance in drug product is above 99% and remains substantially unchanged during storage. These results show that Form CSII drug substance and drug product have good stability under accelerated and stress conditions. Good stability under accelerated and stress conditions is of great importance to the drug development. Drug substance and drug product will go through high temperature and high humidity conditions caused by weather, season and regional climate differences during storage, transportation, and manufacturing processes. Form CSII drug substance and drug product have good stability under these stress conditions, which is beneficial to avoid the influence on drug quality when stored in condition not recommended in the label.

Meanwhile, Form CSII has good mechanical stability. Crystalline state of Form CSII drug substance doesn't change after grinding. Form CSII has good physical stability. Grinding and pulverization are often required in the drug manufacturing process. Good physical stability of the drug substance can reduce the risk of crystallinity decrease and crystal transformation during the drug production process. Form CSII has good physical stability under external pressure, which is beneficial to keep crystalline form unchanged during tableting process.

Crystalline transformation can lead to changes in the absorption of the drug, affect bioavailability, and even cause toxicity and side effects. Good chemical stability ensures that no impurities are generated during storage. Form CSII has good physical and chemical stability, ensuring consistent and controllable quality of the drug substance and drug product, minimizing quality change, bioavailability change and toxicity due to crystal transformation or impurity generation.

Furthermore, Form CSII of the present disclosure also has the following advantages:

(1) Compared with prior art, Form CSII of the present disclosure has uniform particle size distribution. The uniform particle size helps to ensure uniformity of content and reduce variability of in vitro dissolution. At the same time, the preparation process can be simplified, the cost is reduced, and the risk of decrease in crystallinity and crystal transformation caused by grinding can be reduced.

(2) Compared with prior art, Form CSII of the present disclosure has better compressibility. Failure in hardness/friability test and tablet crack issue can be avoided due to better compressibility, making the preparation process more reliable, improving product appearance and product quality. Better compressibility can increase the compression speed, thus further increases the production efficiency and reduces the cost of excipients for improving the compressibility.

(3) Compared with prior art, Form CSII of the present disclosure has a higher yield and is more suitable for industrial production.

(4) Compared with prior art, Form CSII of the present disclosure has better flowability. Flowability evaluation results indicate that the flowability of Form CSII is remarkably better than that of prior art forms. Better flowability can prevent clogging of production equipment and increase manufacturing efficiency. Better flowability of Form CSII ensures the blend uniformity and content uniformity of the drug product, and reduces the weight variation of the drug product and improves product quality.

(5) Compared with prior art, Form CSII of the present disclosure shows superior adhesiveness. Adhesiveness evaluation results indicate that adhesion quantity of Form CSII is remarkably lower than that of prior art forms. Due to the superior adhesiveness of Form CSII, adhesion to roller and tooling during dry-granulation and compression process can be reduced, which is beneficial to improve product appearance and weight variation. In addition, superior adhesiveness of Form CSII can reduce the agglomeration of drug substance and the adhesion of drug substance to the utensil, which is beneficial to the dispersion of drug substance, and improving the blend uniformity and content uniformity of drug product.

According to the objective of the present disclosure, a pharmaceutical composition is provided, said pharmaceutical composition comprises a therapeutically effective amount of Form CSII and pharmaceutically acceptable carriers, diluents or excipients.

Furthermore, Form CSII of the present disclosure can be used for preparing JAK inhibitor drugs.

Furthermore, Form CSII of the present disclosure can be used for preparing drugs treating rheumatoid arthritis, Crohn's disease, ulcerative colitis, atopic dermatitis and psoriatic arthritis.

In the present disclosure, said “stirring” is accomplished by using a conventional method in the field such as magnetic stirring or mechanical stirring and the stirring speed is 50 to 1800 r/min, preferably the magnetic stirring speed is 300 to 900 r/min and mechanical stirring speed is 100 to 300 r/min.

Said “drying” is accomplished at room temperature or a higher temperature. The drying temperature is from room temperature to about 60° C., or to 50° C., or to 40° C. The drying time can be 2-48 hours, or overnight. Drying is carried out in a fume hood, forced air convection oven or vacuum oven.

In the present disclosure, “crystal” or “crystalline form” refers to the crystal or the crystalline form being identified by the X-ray diffraction pattern shown herein. Those skilled in the art are able to understand that physicochemical properties discussed herein can be characterized. The experimental errors depend on the instrument conditions, the sample preparation and the purity of samples. In particular, those skilled in the art generally know that the X-ray diffraction pattern typically varies with the experimental conditions. It is necessary to point out that, the relative intensity of the diffraction peaks in the X-ray diffraction pattern may also vary with the experimental conditions; therefore, the order of the diffraction peak intensities cannot be regarded as the sole or decisive factor. In fact, the relative intensity of the diffraction peaks in the X-ray powder diffraction pattern is related to the preferred orientation of the crystals, and the diffraction peak intensities shown herein are illustrative and identical diffraction peak intensities are not required. In addition, the experimental error of the diffraction peak position is usually 5% or less, and the error of these positions should also be taken into account. An error of ±0.2° is usually allowed. In addition, due to experimental factors such as sample thickness, the overall offset of the diffraction peak is caused, and a certain offset is usually allowed. Thus, it will be understood by those skilled in the art that a crystalline form of the present disclosure is not necessarily to have the exactly same X-ray diffraction pattern of the example shown herein. Any crystalline forms whose X-ray diffraction patterns have the same or similar characteristic peaks should be within the scope of the present disclosure. Those skilled in the art can compare the patterns shown in the present disclosure with that of an unknown crystalline form in order to identify whether these two groups of patterns reflect the same or different crystalline forms.

In some embodiments, Form CSII of the present disclosure is pure and substantially free of any other crystalline forms. In the present disclosure, the term “substantially free” when used to describe a novel crystalline form, it means that the content of other crystalline forms in the novel crystalline form is less than 20% (w/w), specifically less than 10% (w/w), more specifically less than 5% (w/w) and furthermore specifically less than 1% (w/w).

In the present disclosure, the term “about” when referring to a measurable value such as weight, time, temperature, and the like, is meant to encompass variations of ±10%, ±5%, ±1%, 0.5%, or even ±0.1% of the specified amount.

### BRIEF DESCRIPTION OF THE DRAWINGS

FIG. 1 shows an XRPD pattern of Form CSII in Example 1

FIG. 2 shows a TGA curve of Form CSII in Example 1

FIG. 3 shows a DSC curve of Form CSII in Example 1

FIG. 4 shows an XRPD pattern of Form CSII in Example 3

FIG. 5 shows a DSC curve of Form CSII in Example 3

FIG. 6 shows an XRPD pattern of Form CSII in Example 4

FIG. 7 shows a TGA curve of Form CSII in Example 4

FIG. 8 shows a DSC curve of Form CSII in Example 4

FIG. 9 shows an XRPD pattern overlay of Form CSII before and after storage (from top to bottom: Initial, stored at 4° C. for three months in closed dish, stored at 25° C./60% RH for three months in open dish, stored at 25° C./60% for three months in closed dish, stored at 40° C./75% RH for three months in open dish, stored at 40° C./75% RH for three months in closed dish, stored at 60° C./75% RH for one month in open dish, stored at 60° C./75% RH for one month in closed dish)

FIG. 10 shows an XRPD pattern overlay of Form CSII before and after tableting (from top to bottom: after tableting under 10 KN, before tableting)

FIG. 11 shows an XRPD pattern overlay of Form CSII before and after manual grinding (from top to bottom: after grinding; before grinding)

FIG. 12 shows a PSD plot of Form CSII

FIG. 13 shows a PSD plot of Form C in WO2017066775A1

FIG. 14 shows an XRPD pattern overlay of Form CSII before and after preparation into drug product (from top to bottom: Form CSII drug product, excipient blend, Form CSII)

FIG. 15 shows an XRPD pattern overlay of Form CSII drug product from stability test (from top to bottom: Initial, stored at 25° C./60% RH for three months, stored at 40° C./75% RH for three months)

### DETAILED DESCRIPTION OF PREFERRED EMBODIMENTS

The present disclosure is further illustrated by the following examples which describe the preparation and use of the crystalline form of the present disclosure in detail. It is obvious to those skilled in the art that many changes in the materials and methods can be accomplished without departing from the scope of the present disclosure.

The abbreviations used in the present disclosure are explained as follows:

XRPD: X-Ray Powder Diffraction

DSC: Differential Scanning Calorimetry

TGA: Thermo Gravimetric Analysis

¹H NMR: Proton Nuclear Magnetic Resonance

HPLC: High Performance Liquid Chromatography

PSD: Particle Size Distribution

Instruments and methods used for data collection:

X-ray powder diffraction patterns in the present disclosure were acquired by a Bruker D2 PHASER or Bruker D8 Discover X-ray powder diffractometer. The parameters of the X-ray powder diffraction method of the present disclosure are as follows:

X-ray Reflection: Cu, Kα

Kα1 (Å): 1.54060; Kα2 (Å): 1.54439

Kα2/Kα1 intensity ratio: 0.50

Single crystal X-ray diffraction in the present disclosure was acquired by a BRUKER D8 VENTURE X-ray diffractometer. The parameters of the single crystal X-ray diffraction of the present disclosure are as follows:

| | |
|---|---|
| X-Ray Source | IμS microfocus Mo X-ray source, λ = 0.71073 Å |
| Testing temperature | 300 K |
| Detector | PHOTON CMOS Detector |
| Goniometer | FIXED-CHI Goniometer |
| Software package | APEX3 |

Differential scanning calorimetry (DSC) data in the present disclosure were acquired by a TA Q2000. The parameters of the DSC method of the present disclosure are as follows:

Heating rate: 10° C./min

Purge gas: nitrogen

Thermo gravimetric analysis (TGA) data in the present disclosure were acquired by a TA Q500. The parameters of the TGA method of the present disclosure are as follows:

Heating rate: 10° C./min

Purge gas: nitrogen

Dynamic Vapor Sorption (DVS) is measured via an SMS (Surface Measurement Systems Ltd.) intrinsic DVS instrument. Its control software is DVS-Intrinsic control software. Typical Parameters for DVS test are as follows:

Temperature: 25° C.

Gas and flow rate: N₂, 200 mL/min

dm/dt: 0.002%/min

RH range: 0% RH to 95% RH

Proton nuclear magnetic resonance (H NMR) spectrum data were collected from a Bruker Avance II DMX 400M HZ NMR spectrometer. 1-5 mg of sample was weighed and dissolved in 0.5 mL of deuterated dimethyl sulfoxide to obtain a solution with a concentration of 2-10 mg/mL. The particle size distribution data in the present disclosure were acquired by a Mastersizer 3000 laser particle size analyzer of Malvern. The test is carried out in wet mode with a Hydro MV dispersing device, and the dispersion medium is Isopar G. The method parameters of the laser particle size analyzer are as follows:

| | |
|---|---|
| Size distribution: Volume | Run Time: 10 s |
| Dispersion medium: Isopar G | Particle coordinates: Standard |
| Run Number: 3 | Fluid refractive index: 1.42 |
| Absorption rate: 0.100 | Residuals: Enabled |
| Particle refractive index: 1.520 | Rotational speed: 2500 rpm |
| Particle shape: Irregular | Ultrasonication power/time: 30 W/30 s |

The method parameters of kinetic solubility test in the present disclosure are as follows:

| HPLC | Agilent 1260 with Variable Wavelength Detector | |
|---|---|---|
| | (VWD) | |
| Column | Waters Xbridge C18, 150*4.6 mm, 5 μm | |
| Mobile phase | A: 0.1% trifluoroacetic acid in water | |
| | B: 0.1% trifluoroacetic acid in acetonitrile | |
| Gradient | Time (min) | % B |
| | 0.0 | 20.0 |
| | 10.0 | 20.0 |

| | |
|---|---|
| Running time | 10.0 min |
| Equilibration time | 0.0 min |
| Flow rate | 1.0 mL/min |
| Injection volume | 5 μL |
| Detection wavelength | UV at 220 nm |
| Column Temperature | 40° C. |
| Temperature of | Room Temperature |
| sample tray | |
| Diluent | 50% acetonitrile aqueous solution |

The method parameters for relative substances test in the present disclosure are as follows:

| HPLC | Agilent 1260 with VWD | |
|---|---|---|
| Column | L033 phenomenex Gemini C18 110A, 250 × 4.6 | |
| | mm, 3 μm | |
| Mobile phase | A: 10 mM KH₂PO₄ aqueous solution (pH = 2.5) | |
| | B: Acetonitrile | |
| Gradient | Time (min) | % B |
| | 0.00 | 5 |
| | 3.00 | 5 |
| | 40.00 | 80 |
| | 45.00 | 80 |
| | 46.00 | 5 |
| | 55.00 | 5 |

| | |
|---|---|
| Running time | 55 min |
| Flow rate | 1.0 mL/min |
| Injection volume | 3 μL |
| Detection wavelength | UV at 210 nm |
| Column temperature | 40° C. |
| Temperature of | Room Temperature |
| sample tray | |
| Diluent | 50% acetonitrile aqueous solution |

The method parameters for drug products dissolution profile measurement in the present disclosure are as follows:

| HPLC | Agilent 1260 with Diode Array Detector | |
|---|---|---|
| | (DAD) | |
| Column | Waters XBridge C18, 150*4.6 mm, 5 μm | |
| Mobile phase | A: 0.1% trifluoroacetic acid in water | |
| | B: 0.1% trifluoroacetic acid in acetonitrile | |
| Gradient | Time (min) | % B |
| | 0.0 | 20 |
| | 10.0 | 20 |

| | |
|---|---|
| Running time | 10.0 min |
| Flow rate | 1.0 mL/min |
| Injection Volume | 5 μL |
| Detection wavelength | UV at 220 nm |
| Column temperature | 40° C. |
| Temperature of | Room Temperature |
| sample tray | |
| Diluent | 50% acetonitrile aqueous solution |

Unless otherwise specified, the following examples were conducted at room temperature. Said “room temperature” is not a specific temperature, but a temperature range of 10−30° C.

According to the present disclosure, upadacitinib and/or its salt used as a raw material is solid (crystalline or amorphous), oil, liquid form or solution. Preferably, compound I and/or its salt used as a raw material is a solid.

Upadacitinib and/or a salt thereof used in the following examples were prepared by known methods, for example, the method disclosed in WO2017066775A1. Form C of WO2017066775A1 in the present disclosure was prepared by method A of example 7 disclosed in WO2017066775A1.

### Example 1 Preparation of Form CSII

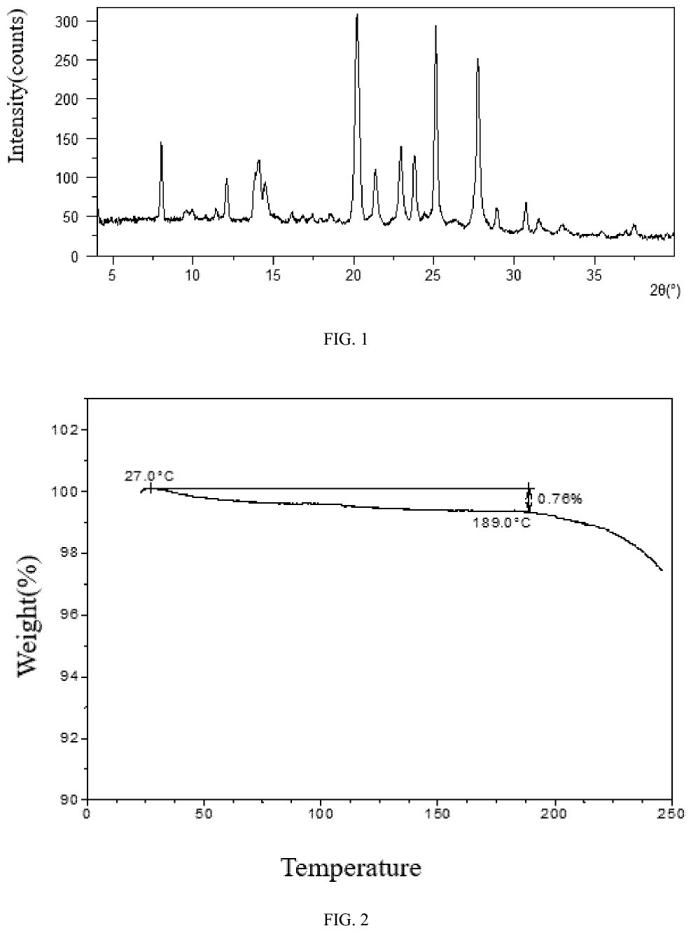

49.2 mg of upadacitinib free base amorphous was weighed into a 5-mL glass vial followed by adding 5.0 mL of water saturated isopropyl ether/isopropyl ether (2:1, v/v) to form a suspension. The suspension was transferred to a hot stage (80° C.), heated for about 5 h, placed in a fume hood and kept at room temperature for about 23 h. Then the suspension was transferred to a hot stage (80° C.), heated for about 4 h, then placed in a fume hood and kept at room temperature for about 12 h. Then the suspension was transferred to 80° C. hot stage for about 96 h. A solid was obtained by isolation and drying at room temperature. The solid was confirmed to be Form CSII of the present disclosure. The XRPD pattern is substantially as depicted in FIG. 1, and the XRPD data are listed in Table 1.

The TGA curve of Form CSII in the present disclosure shows about 0.8% weight loss when heated to 189° C., which is substantially as depicted in FIG. 2. The weight loss corresponds to the loss of a small amount of adsorbed water and isopropyl ether.

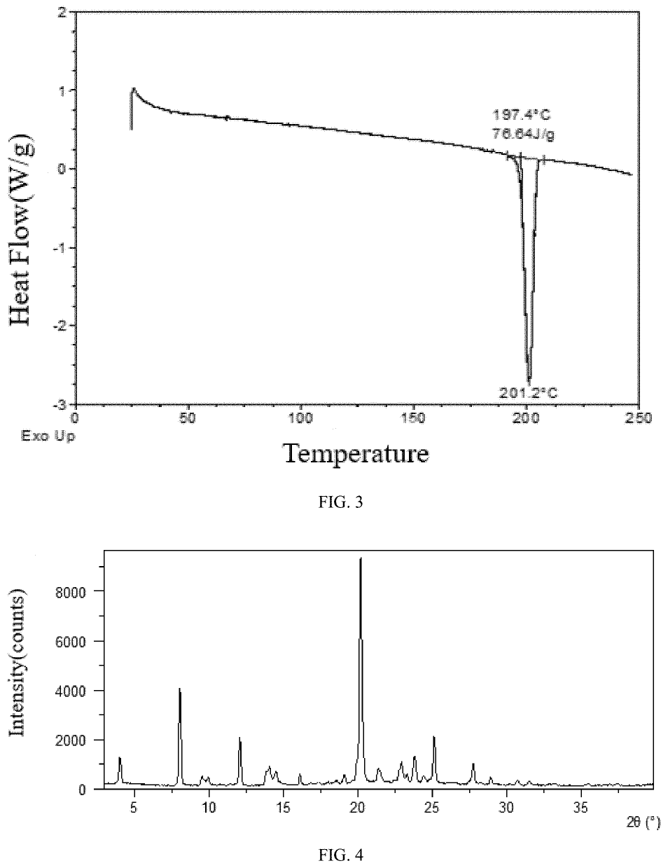

The DSC curve of Form CSII in the present disclosure is substantially as depicted in FIG. 3, which shows one endothermic peak at around 197° C. corresponding to the melting endothermic peak.

Without any limitation being implied, Form CSII is an anhydrate.

TABLE 1

| 2θ | d spacing | Intensity % |
|---|---|---|
| 8.04 | 11.00 | 37.23 |
| 12.09 | 7.32 | 19.67 |
| 14.02 | 6.32 | 27.24 |
| 20.18 | 4.40 | 100.00 |
| 21.33 | 4.17 | 26.93 |
| 22.95 | 3.88 | 39.78 |
| 23.77 | 3.74 | 34.92 |
| 25.11 | 3.55 | 98.68 |
| 27.74 | 3.22 | 84.95 |
| 28.92 | 3.09 | 10.78 |
| 30.74 | 2.91 | 13.62 |
| 37.54 | 2.40 | 4.66 |

### Example 2 Preparation of Form CSII

4.3 mg of upadacitinib free base amorphous was weighed into a 3-mL glass vial followed by adding 2.0 mL of isopropyl ether to form a suspension. The suspension was treated by ultrasonication at room temperature for 30 s and then placed at 50° C. for about 24 h. Light yellow solid was obtained by isolation and drying at room temperature. The obtained solid was confirmed to be Form CSII by XRPD.

### Example 3 Preparation of Form CSII

As shown in Table 2, about 50 mg of upadacitinib free base amorphous was weighed into a 5-mL glass vial followed by adding 5.0 mL of water saturated isopropyl ether/isopropyl ether (2:1, v/v) to form a suspension. The suspension was treated by ultrasonication at room temperature for 1 min and then placed in a 50° C. oven for about 66 h. Then the suspension was placed in a fume hood at room temperature for about 202 h, transferred to an oven at 50° C. for about 20 h, and placed in a fume hood at room temperature for about 2 h. The suspension was transferred to an oven at 50° C. for about 16 h, and placed in a fume hood at room temperature for about 26 h. A solid was obtained by isolation. The solid in 32 glass vials was combined and dried under vacuum at 25° C. for about 3 h, and then the solid was mixed manually for 2 min. The solid was confirmed to be Form CSII. The XRPD pattern is substantially as depicted in FIG. 4 and the XRPD data are listed in Table 3.

TABLE 2

| Number | Weight (mg) |
|---|---|
| 1 | 50.8 |
| 2 | 49.8 |
| 3 | 48.8 |
| 4 | 49.8 |
| 5 | 48.0 |
| 6 | 49.3 |
| 7 | 49.4 |
| 8 | 50.4 |
| 9 | 48.9 |
| 10 | 50.6 |
| 11 | 51.0 |
| 12 | 50.5 |
| 13 | 48.3 |
| 14 | 51.2 |
| 15 | 49.6 |
| 16 | 50.6 |
| 17 | 47.0 |
| 18 | 47.5 |
| 19 | 49.7 |
| 20 | 50.3 |
| 21 | 50.2 |
| 22 | 50.4 |
| 23 | 49.5 |
| 24 | 50.2 |
| 25 | 50.6 |
| 26 | 50.0 |
| 27 | 47.9 |
| 28 | 47.4 |
| 29 | 52.0 |
| 30 | 50.8 |
| 31 | 51.5 |
| 32 | 49.2 |

The DSC curve is substantially as depicted in FIG. 5, which shows an endothermic peak at around 196° C., corresponding to the melting endothermic peak.

The ¹H NMR data are consistent with the compound structure, and the corresponding data are: ¹H NMR (400 MHz, DMSO) δ 12.28 (s, 1H), 8.58 (s, 1H), 7.48 (s, 1H), 7.45 (t, J=3.1 Hz, 1H), 7.01 (dd, J=3.3, 2.0 Hz, 1H), 6.97 (t, J=6.3 Hz, 1H), 4.36 (dd, J=6.2, 6.2 Hz, 1H), 3.95-3.73 (m, 4H), 3.69 (dd, J=10.2, 6.9 Hz, 1H), 3.27 (dd, J=10.2, 6.1 Hz, 1H), 2.57 (dt, J=10.5, 5.3 Hz, 1H), 1.18-1.05 (m, 1H), 0.90-0.74 (m, 1H), 0.64 (t, J=7.4 Hz, 3H).

TABLE 3

| 2θ | d spacing | Intensity % |
|---|---|---|
| 4.01 | 22.06 | 12.06 |
| 8.02 | 11.03 | 44.17 |
| 9.50 | 9.31 | 4.32 |
| 9.95 | 8.89 | 3.55 |
| 12.06 | 7.34 | 22.49 |
| 13.84 | 6.40 | 5.91 |
| 14.06 | 6.30 | 8.70 |
| 14.53 | 6.10 | 6.28 |
| 16.10 | 5.50 | 4.88 |
| 17.38 | 5.10 | 1.61 |
| 18.54 | 4.78 | 2.16 |
| 19.08 | 4.65 | 5.19 |
| 19.92 | 4.46 | 11.30 |
| 20.17 | 4.40 | 100.00 |
| 21.36 | 4.16 | 7.85 |
| 22.95 | 3.87 | 10.82 |
| 23.32 | 3.81 | 5.59 |
| 23.82 | 3.74 | 13.85 |
| 24.43 | 3.64 | 4.35 |
| 25.11 | 3.55 | 21.71 |
| 26.37 | 3.38 | 1.44 |
| 27.80 | 3.21 | 9.75 |
| 28.95 | 3.08 | 3.83 |
| 30.74 | 2.91 | 2.33 |
| 31.50 | 2.84 | 2.01 |
| 33.23 | 2.70 | 0.71 |
| 35.46 | 2.53 | 0.77 |
| 37.47 | 2.40 | 1.08 |

### Example 4 Preparation of Form CSII

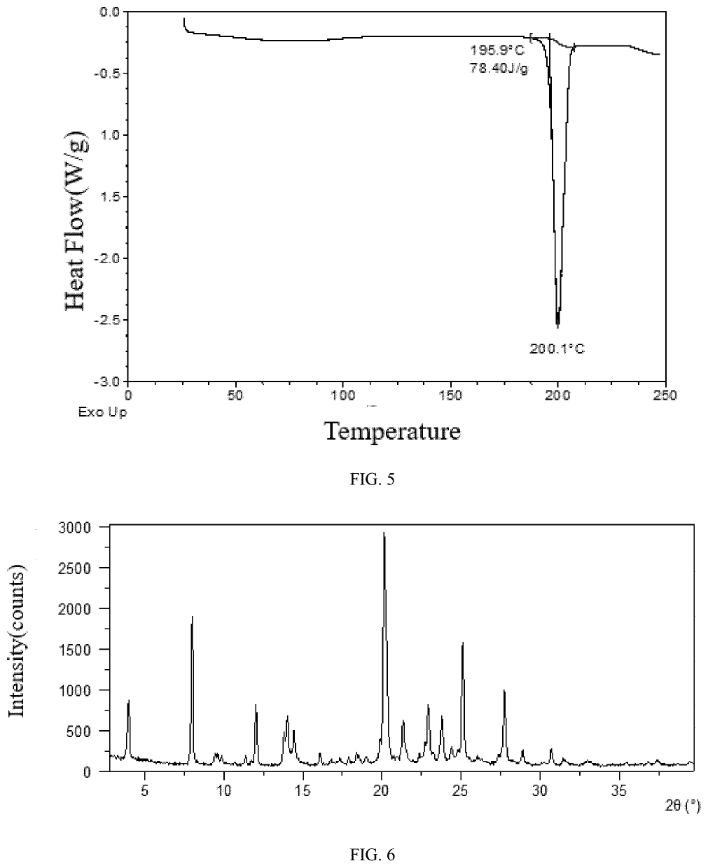

As shown in Table 4, about 50 mg of upadacitinib free base amorphous was weighed into a 5-mL glass vial followed by adding 5.0 mL of a solvent mixture comprising isopropyl ether solution saturated with water and isopropyl ether (2:1, v/v) to form a suspension. The suspension was treated by ultrasonication at room temperature for 1 min and put to an oven at 50° C. for about 70 h. Then the suspension was placed in a fume hood at room temperature for about 68 h. A solid was obtained by isolation. The solid in 22 glass vials was combined and dried under vacuum at 25° C. for about 2 h. The solid obtained was confirmed to be Form CSII. The XRPD pattern is substantially as depicted in FIG. 6 and the XRPD data are listed in Table 5.

TABLE 4

| Number | Weight (mg) |
|---|---|
| 1 | 48.9 |
| 2 | 50.4 |
| 3 | 48.4 |
| 4 | 48.8 |
| 5 | 48.3 |
| 6 | 51.6 |
| 7 | 48.3 |
| 8 | 50.1 |
| 9 | 50.2 |
| 10 | 47.3 |
| 11 | 49.5 |
| 12 | 47.3 |
| 13 | 50.2 |
| 14 | 50.3 |
| 15 | 49.7 |
| 16 | 48.2 |
| 17 | 52.2 |
| 18 | 48.9 |
| 19 | 49.0 |
| 20 | 49.1 |
| 21 | 50.5 |
| 22 | 48.0 |

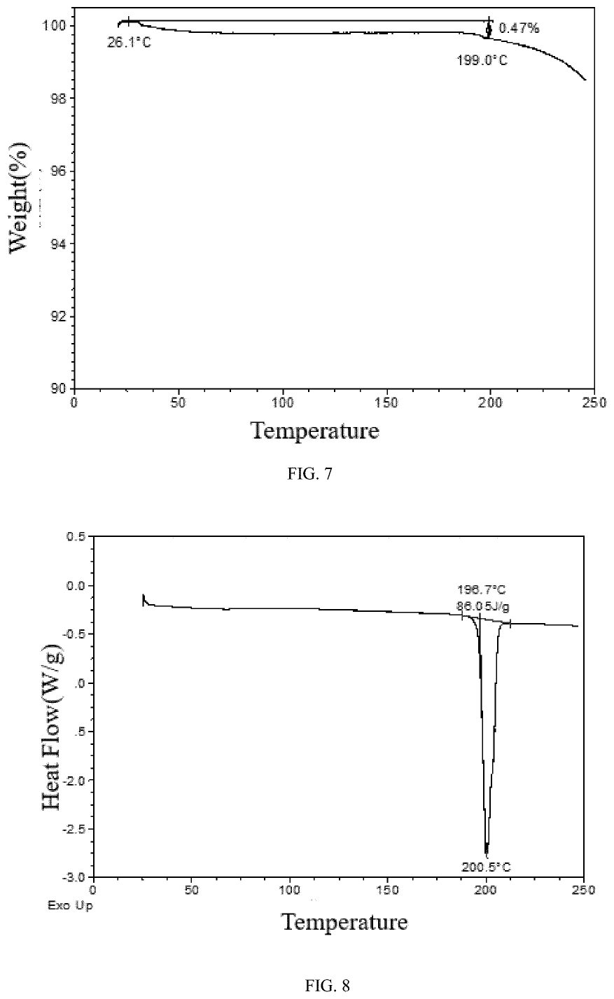

The TGA curve shows about 0.5% weight loss when heated to 199° C., which is substantially as depicted in FIG. 7. The weight loss corresponds to the removal of the residual solvent.

The DSC curve is substantially as depicted in FIG. 8, which shows one endothermic peak at around 197° C., corresponding to the melting endothermic peak.

TABLE 5

| 2θ | d spacing | Intensity % |
|---|---|---|
| 4.02 | 22.00 | 26.90 |
| 8.02 | 11.03 | 66.24 |
| 9.44 | 9.37 | 4.33 |
| 9.87 | 8.96 | 3.82 |
| 11.38 | 7.77 | 4.03 |
| 12.04 | 7.35 | 27.13 |
| 13.79 | 6.42 | 13.48 |
| 14.06 | 6.30 | 19.11 |
| 14.44 | 6.14 | 15.63 |
| 16.07 | 5.52 | 5.01 |
| 17.34 | 5.11 | 2.81 |
| 17.90 | 4.95 | 3.36 |
| 18.48 | 4.80 | 4.57 |
| 19.02 | 4.67 | 4.02 |
| 20.14 | 4.41 | 100.00 |
| 21.35 | 4.16 | 20.18 |
| 22.92 | 3.88 | 26.55 |
| 23.81 | 3.74 | 21.70 |
| 24.43 | 3.64 | 8.69 |
| 25.11 | 3.55 | 55.13 |
| 26.24 | 3.40 | 2.70 |
| 27.72 | 3.22 | 33.52 |
| 28.91 | 3.09 | 6.00 |
| 30.69 | 2.91 | 7.05 |
| 31.53 | 2.84 | 2.56 |
| 33.05 | 2.71 | 1.92 |
| 37.42 | 2.40 | 1.97 |

### Example 5 Single Crystal of Form CSII

66.2 mg of upadacitinib free base amorphous was weighed into a 20-mL glass vial followed by adding 3 mL of dimethyl carbonate to obtain a solution. 1 mL of the obtained solution was filtrated into an HPLC glass vial and 2-5 mg of Mixture B (Mixture B comprises the following substances by equal weight: PCL (polycaprolactone), PEG (polyethylene glycol), PMMA (polymethyl methacrylate), SA (stearyl acrylate) and HEC (hydroxyethyl cellulose)) was added. The system was evaporated at room temperature for about 6 days. Crystalline solid was collected and tested by an X-ray single crystal diffractometer. The single crystal structure of Form CSII was obtained by single crystal determination. The single crystal data of Form CSII are listed in Table 6.

TABLE 6
Single Crystal Structure Parameters

| | |
|---|---|
| Crystal system | Triclinic crystal system |
| Space group | P1 |
| Unit cell dimensions | a = 8.706(7) Å |
| | b = 9.397(8) Å |
| | c = 22.189(18) Å |
| | α = 96.66(3)° |
| | β = 97.31(2)° |
| | γ = 90.48(3)° |
| Volume of unit cell (V) | 1787.89 Å³ |
| Number of formula units in | 4 |
| unit cell (Z) | |

### Example 6 Kinetic Solubility of Form CSII

The solubility of Form C is disclosed in WO2017066775A1. To compare with Form C, solubility of Form CSII was measured. A certain amount of Form CSII was suspended into pH=7.4 PBS, pH=6.5 FaSSIF and pH=5.0 FeSSIF at 25° C. or 37° C. to obtain saturated solutions. After equilibration for 24 h, 30 h and 48 h, the suspensions were filtered to obtain saturated solutions, and concentration was determined by HPLC. The results are listed in Table 7.

TABLE 7

| | Solubility | | | |
|---|---|---|---|---|
| | Form CSII | | | |
| | 24 h | 30 h | 48 h | Form C |
| Media | (mg/mL) | (mg/mL) | (mg/mL) | (mg/mL) |
| PBS, 25° C. | 0.73 | 0.74 | 0.72 | 0.19 |
| FaSSIF, (37° C.) | 0.74 | 0.76 | 0.83 | 0.22 |
| FeSSIF, (37° C.) | 1.4 | 1.4 | 1.5 | 0.47 |

The results show that Form CSII has a higher solubility in pH=7.4 PBS, FaSSIF and FeSSIF.

### Example 7 Stability of Form CSII

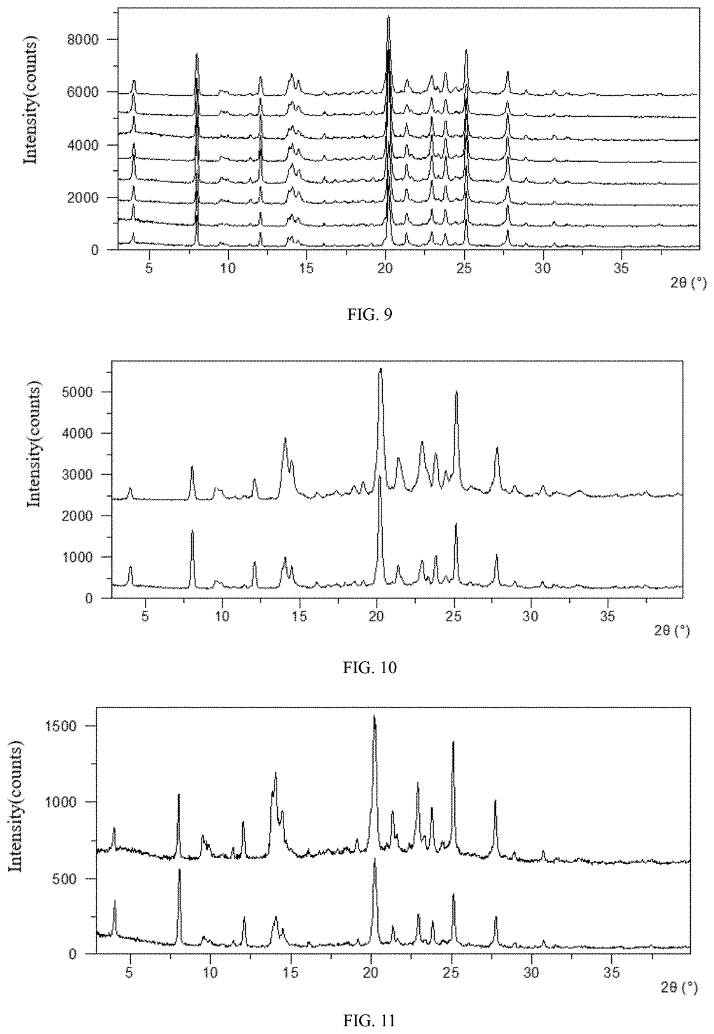

A certain amount of Form CSII sample was stored at different conditions of 4° C., 25° C./60% RH, 40° C./75% RH and 60° C./75% RH. Crystalline form was checked by XRPD before and after storage. The results are shown in Table 8, and the XRPD overlay is shown in FIG. 9.

TABLE 8

| Condition | | Storage time | | Solid form | Purity |
|---|---|---|---|---|---|
| Initial solid form | | — | | Form CSII | 99.35% |
| 4° C. | Closed | 3 | months | Form CSII | 99.41% |
| 25° C./60% RH | Open | 3 | months | Form CSII | 99.43% |
| | Closed | | | Form CSII | 99.42% |
| 40° C./75% RH | Open | 3 | months | Form CSII | 99.44% |
| | Closed | | | Form CSII | 99.42% |
| 60° C./75% RH | Open | 1 | month | Form CSII | 99.48% |
| | Closed | | | Form CSII | 99.43% |

The results show that Form CSII kept stable for at least 3 months at 4° C. and 25° C./60% RH. It shows that Form CSII has good stability under long-term conditions. Form CSII kept stable for at least 3 months at 40° C./75% RH and at least 1 month at 60° C./75% RH. It shows that Form CSII has good stability under more stress conditions.

### Example 8 Mechanical Stability of Form CSII

30 mg of Form CSII was weighed into the dies of a Φ8 mm round tooling and tableted by ENERPAC manual tablet press with 10 kN pressure. Crystalline form before and after tableting were checked by XRPD. The test results are shown in FIG. 10. The results show that crystalline state of Form CSII does not change and the crystallinity of Form CSII also remained basically unchanged after being compressed into a tablet. 10 mg of Form CSII was weighed into a mortar and ground manually for 5 min. The crystalline form before and after grinding were checked by XRPD. The test results are shown in FIG. 11. The results show that crystalline state of Form CSII does not change and the crystallinity of Form CSII also remained basically unchanged after grinding.

### Example 9 Particle Size Distribution of Form CSII

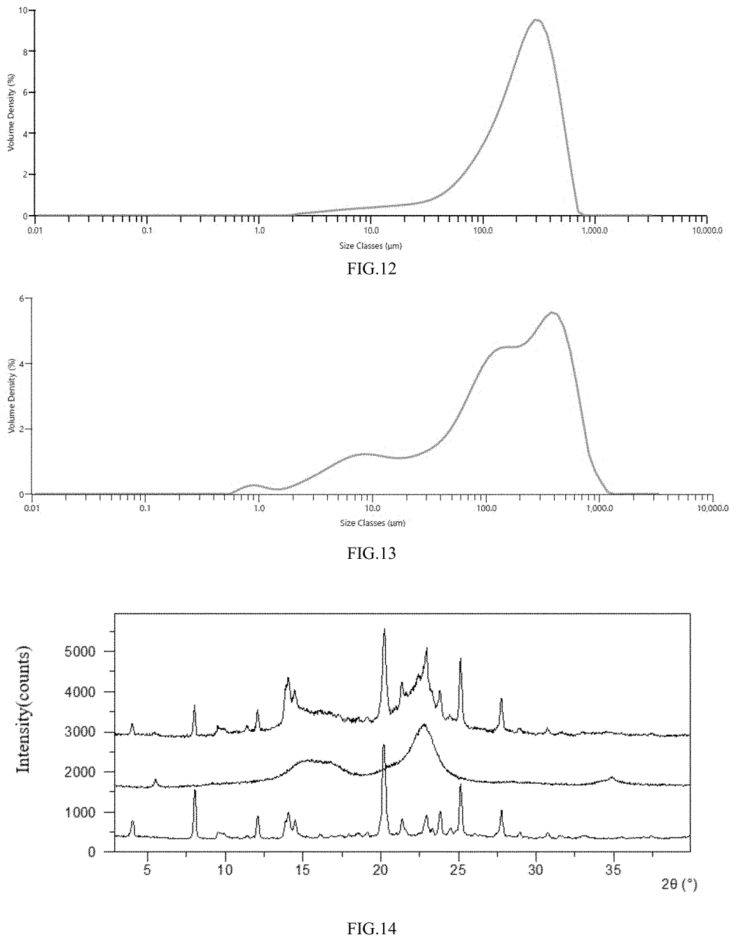

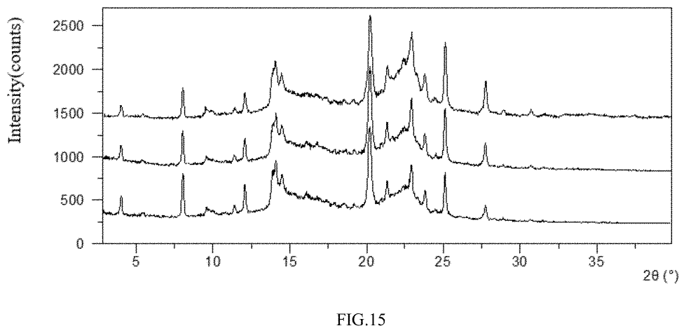

10-30 mg of Form CSII or Form C in WO2017066775A1 was added into glass vials with about 5 mL of Isopar G (containing 0.2% lecithin). The mixtures were mixed thoroughly and transferred into the Hydro MV. The measurement was started when the shading rate is in an appropriate position. The particle size distribution diagrams of Form CSII and Form C are shown in FIG. 12 and FIG. 13. The results show that the particle size distribution of Form CSII is uniform, which is superior to that of WO2017066775A1 Form C.

### Example 10 Yield of Form CSII

Form C in WO2017066775A1: 1.5 g of upadacitinib free base was weighed and dissolved into 47.5 mL of ethanol. The obtained solution was filtered into a 500-mL crystallizer, and then 150 mL of water was added slowly with stirring at 6° C. The system was stirred overnight and 1.13 g solid was isolated. The corresponding yield was 79.0% (in terms of upadacitinib free base).

Form CSII. The yield of Form CSII obtain in Example 4 was 86.4% (in terms of upadacitinib free base).
The results show that the yield of Form CSII is higher than that of Form C in WO2017066775A1.

### Example 11 Flowability of Form CSII

Compressibility index or Carr index is usually utilized to evaluate the flowability of powder and granules during the drug product process. Compressibility index test method is as follows: a certain amount of powder was added into a measuring cylinder and bulk volume was recorded. Then the powder was tapped to make it in the tightest state and the tapped volume was recorded. The bulk density ρ₀ and tapped density pf were calculated. The compressibility index was calculated according to c=(ρ_{f}−ρ₀)/ρ_{f}.

Criteria of flowability according to ICH Q4B Annex 13 is shown in Table 9.

TABLE 9

| Compressibility index (%) | Flowability |
|---|---|
| ≤10 | Excellent |
| 11-15 | Good |
| 16-20 | Fair |
| 21-25 | Passable |
| 26-31 | poor |
| 32-37 | Very poor |
| >38 | Very, very poor |

Equipment: ZS-2E tap density tester.

Parameter: 5-mL measuring cylinder, 500 mg, tapping times: 1250.

Flowability evaluation results of Form CSII and Form C in WO2017066775A1 are presented in Table 10, which indicate that flowability of Form CSII is remarkably superior to that of Form C in prior art.

TABLE 10

| | Bulk | Tapped | Compress- | |
|---|---|---|---|---|
| | density | density | ibility | Flow- |
| Solid form | (ρ₀, g/mL) | (ρ_{f}, g/mL) | index (%) | ability |
| Form C in | 0.3611 | 0.5618 | 36% | Very poor |
| WO2017066775A1 | | | | |
| Form CSII | 0.3295 | 0.3548 | 7% | Excellent |

### Example 12 Compressibility of Form CSII

An ENERPAC manual tablet press was used for compression. 80 mg of Form CSII and Form C in the prior art were weighed and added into the dies of a Φ6 mm round tooling, compressed at 10 kN manually, then stored at room temperature for 24 h. Until complete elastic recovery, hardness (H) was tested with an intelligent tablet hardness tester. Diameter (D) and thickness (L) were tested with a caliper. Tensile strength of the powder was calculated with the following formula: T=2H/πDL. Under a certain force, the greater the tensile strength, the better the compressibility. The results are presented in Table 11 and Table 12.

TABLE 11
Form CSII

| Number | 1 | 2 | 3 |
|---|---|---|---|
| Thickness (mm) | 2.35 | 2.16 | 2.12 |
| Diameter (mm) | 6.05 | 6.04 | 6.03 |
| Hardness (kgf) | 3.02 | 1.03 | 1.65 |
| Tensile strength (MPa) | 1.33 | 0.50 | 0.81 |

| | |
|---|---|
| Average tensile strength (MPa) | 0.88 |

TABLE 12
Form C in WO2017066775A1

| Number | 1 | 2 | 3 |
|---|---|---|---|
| Thickness (mm) | 2.14 | 2.18 | 2.07 |
| Diameter (mm) | 6.00 | 6.01 | 6.00 |
| Hardness (kgf) | 0.84 | 1.70 | 1.70 |
| Tensile strength (MPa) | 0.41 | 0.81 | 0.85 |

| | |
|---|---|
| Average tensile strength (MPa) | 0.69 |

The results indicate that Form CSII has better compressibility compared with Form C in WO2017066775A1.

### Example 13 Adhesiveness of Form CSII

30 mg of Form CSII and Form C in WO2017066775A1 were weighed and added into the dies of a Φ8 mm round tooling, compressed at 10 kN and held for 30 s. The amount of material sticking to the punch was weighed. The compression was repeated twice and the average amount of material sticking to the punch during the compression were recorded. Detailed experimental results are shown in Table 13.

TABLE 13

| Solid form | Average amount (mg) |
|---|---|
| Form C in WO2017066775A1 | 0.21 |
| Form CSII | 0.16 |

The results indicate that the amount sticking to the punch of Form C in the prior art is more than hat of Form CSII. The adhesiveness of Form CSII is superior to that of Form C in prior art.

### Example 14 Preparation of CSII Drug Product

Form CSII of the present disclosure was made into tablets by using the formulation and process listed in Table 14 and Table 15. Crystalline form before and after tableting were checked by XRPD. The results show that Form CSII was stable in the formulation process the formulation process.

TABLE 14

| | | | | |
|---|---|---|---|---|
| | | Quantity | % | |
| No. | Component | (mg/unit) | (w/w) | Function |

Intra-granular material

| | | | | |
|---|---|---|---|---|
| 1 | Upadacitinib free base | 30.0 | 30.0 | API |
| 2 | Microcrystalline cellulose | 60.0 | 60.0 | Filler |
| | (PH 101) | | | |
| 3 | Hypromellose (E5) | 3.0 | 3.0 | Binder |
| 4 | Crospovidone (XL) | 6.0 | 6.0 | Disintegrant |
| 5 | Magnesium stearate (5712) | 0.5 | 0.5 | Lubricant |

Extra-granular material

| | | | | |
|---|---|---|---|---|
| 6 | Magnesium stearate (5712) | 0.5 | 0.5 | Lubricant |

| | | | |
|---|---|---|---|
| Total | 100.00 | 100.00 | N/A |

TABLE 15

| Stage | Procedure |
|---|---|
| Pre-blending | According to the formulation, materials No. 1-5were |
| | weighted into a PE (polyethylene) bag and blended |
| | manually for 2 min. |
| Sifting | The mixture was sieved through a 35 mesh sieves and |
| | then put in a PE bag and mixed for 1 min. |
| Simulated | The tablets were prepared by compressing with a |
| dry | manual single punch tablet press. (type: ENERPAC; die: |
| granulation | φ 20 mm round; tablet weight: 500 mg ± 10.0 mg; |
| | pressure: 5 ± 1 KN) |
| Mill | The tablets were crushed into the granules, then |
| | passed through a 20-mesh sieve. |
| Final | The extra-granular excipient and sieved granules |
| blending | were blended manually for 2 min in a PE bag. |
| Compression | The tablets were prepared by compressing with a single |
| | punch manual tablet press. (Model: ENERPAC; Die: φ7 |
| | mm round; Weight: 100.0 mg ± 2.0 mg; Pressure: 5 |
| | kN ± 1 kN) |
| Package | The tablets were packed in 35 cc HDPE (High density |
| | polyethylene) bottles, one tablet per bottle with 1 g |
| | of desiccant. |

### Example 15 Stability of Form CSII in Drug Product

The drug products of Form CSII were stored under 25° C./60% RH and 40° C./75% RH in closed dishes with 1 g of desiccant for 3 months to evaluate the stability of Form CSII in drug product. The results are shown in Table 16. XRPD patterns before and after storage are shown in FIG. 15. The results indicate that Form CSII drug product is physically and chemically stable under 25° C./60% RH and 40° C./75% RH for at least 3 months.

TABLE 16

| Condition | Time | Solid form | Purity % |
|---|---|---|---|
| Initial | — | Form CSII | 99.38 |
| 25° C./60% RH in closed dish | 3 months | Form CSII | 99.37 |
| 40° C./75% RH in closed dish | 3 months | Form CSII | 99.40 |

The examples described above are only for illustrating the technical concepts and features of the present disclosure and intended to make those skilled in the art being able to understand the present disclosure and thereby implement it, and should not be concluded to limit the protective scope of this disclosure. Any equivalent variations or modifications according to the spirit of the present disclosure should be covered by the protective scope of the present disclosure.

1. A crystalline form CSII of upadacitinib, wherein:
(1) the X-ray powder diffraction pattern shows characteristic peaks at 2theta values of 20.2°±0.2°, 25.1°±0.2° and 27.7°±0.2° using CuKα radiation; or
(2) the crystalline form CSII has the following single crystal structure parameters:
Crystal system: triclinic,
Space group: P1,
Unit cell parameters: a=8.706(7) Å, b=9.397(8) Å, c=22.189(18) Å, α=96.66(3)°, β=97.31(2)°, γ=90.48(3)°.

2. The crystalline form CSII according to claim 1, wherein the X-ray powder diffraction pattern shows one or two or three characteristic peaks at 2theta values of 8.0°±0.2°, 23.0°±0.2° and 23.8°±0.2° using CuKα radiation.

3. The crystalline form CSII according to claim 1, wherein the X-ray powder diffraction pattern shows one or two characteristic peaks at 2theta values of 21.3°±0.2° and 12.1°±0.2° using CuKα radiation.

4. A process for preparing crystalline form CSII of upadacitinib according to claim 1, wherein the process comprises:
dispersing upadacitinib free base in an ether solvent, keeping the system at 10-100° C. for reaction to obtain crystalline form CSII.

5. The process according to claim 4, wherein said ether solvent is R1-O—R2 or a mixture thereof, R1 and R2 are C2-C5 short-chain alkyls.

6. The process according to claim 5, wherein said ether is isopropyl ether.

7. A pharmaceutical composition, wherein the pharmaceutical composition comprises a therapeutically effective amount of crystalline form CSII and pharmaceutically acceptable carriers, diluents or excipients.

8. (canceled)

9. A method of treating a disease or condition selected from the group consisting of rheumatoid arthritis, Crohn's disease, ulcerative colitis, atopic dermatitis and psoriatic arthritis comprising administering to a subject in need thereof a therapeutically effective amount of crystalline form CSII according to claim 1.
